SEQUENCE LISTING (41 sequences)

SEQ ID NO: 1 - protein, 108 aa, Artificial Sequence
  light variable chain of modified trastuzumab scFv
DIQMTQSPSSLSASVGDRVTITCRASQDVKRAVAWYQQKPGKAPKLLIYWASFLYSGVPSRFSGSRSGTDFTLTISSLQPEDFATYYCQQFYTTPPTFGQGTKVEIKR

SEQ ID NO: 2 - protein, 108 aa, Artificial Sequence
  light variable chain of modified trastuzumab scFv
DIQMTQSPSSLSASVGDRVTITCRASQDVQTAVAWYQQKPGKAPKLLIYSASFLYSGVPSRFSGSRSGTDFTLTISSLQPEDFATYYCQQHYTTPPTFGQGTKVEIKR

SEQ ID NO: 3 - protein, 108 aa, Artificial Sequence
  light variable chain of modified trastuzumab scFv
DIQMTQSPSSLSASVGDRVTITCRASQDVLRAVAWYQQKPGKAPKLLIYLASFLYSGVPSRFSGSRSGTDFTLTISSLQPEDFATYYCQQFWTTPPTFGQGTKVEIKR

SEQ ID NO: 4 - protein, 108 aa, Artificial Sequence
  light variable chain of modified trastuzumab scFv
DIQMTQSPSSLSASVGDRVTITCRASQKVGVAVAWYQQKPGKAPKLLIYWAEFRYDGVPSRFSGSESGTDFTLTISSLQPEDFATYYCQQFYDTPPTFGQGTKVEIKR

SEQ ID NO: 5 - protein, 108 aa, Artificial Sequence
  light variable chain of modified trastuzumab scFv
DIQMTQSPSSLSASVGDRVTITCRASQKVGTAVAWYQQKPGKAPKLLIYSAEFLYSGVPSRFSGSESGTDFTLTISSLQPEDFATYYCQQHYDTPPTFGQGTKVEIKR

SEQ ID NO: 6 - protein, 108 aa, Artificial Sequence
  light variable chain of modified trastuzumab scFv
DIQMTQSPSSLSASVGDRVTITCRASQKVGTAVAWYQQKPGKAPKLLIYDAEFLYSGVPSRFSGSESGTDFTLTISSLQPEDFATYYCQQHYDTPPTFGQGTKVEIKR

SEQ ID NO: 7 - protein, 108 aa, Artificial Sequence; identical to SEQ ID NO: 3
  light variable chain of modified trastuzumab scFv

SEQ ID NO: 8 - protein, 108 aa, Artificial Sequence; identical to SEQ ID NO: 3
  light variable chain of modified trastuzumab scFv

SEQ ID NO: 9 - protein, 108 aa, Artificial Sequence; identical to SEQ ID NO: 3
  light variable chain of modified trastuzumab scFv

SEQ ID NO: 10 - protein, 108 aa, Artificial Sequence
  light variable chain of modified trastuzumab scFv
DIQMTQSPSSLSASVGDRVTITCRASQKVGVAVAWYQQKPGKAPKLLIYWASFLYDGVPSRFSGSGSGTDFTLTISSLQPEDFATYYCQQFYDTPPTFGQGTKVEIKR

SEQ ID NO: 11 - protein, 108 aa, Artificial Sequence
  light variable chain of modified trastuzumab scFv
DIQMTQSPSSLSASVGDRVTITCRASQKVGTAVAWYQQKPGKAPKLLIYSASFLYSGVPSRFSGSGSGTDFTLTISSLQPEDFATYYCQQHYDTPPTFGQGTKVEIKR

SEQ ID NO: 12 - protein, 108 aa, Artificial Sequence
  light variable chain of modified trastuzumab scFv
DIQMTQSPSSLSASVGDRVTITCRASQKVGTAVAWYQQKPGKAPKLLIYDASFLYSGVPSRFSGSGSGTDFTLTISSLQPEDFATYYCQQHYDTPPTFGQGTKVEIKR

SEQ ID NO: 13 - protein, 108 aa, Artificial Sequence
  light variable chain of modified trastuzumab scFv
DIQMTQSPSSLSASVGDRVTITCRASQKVGTAVAWYQQKPGKAPKLLIYWAEFLYSGVPSRFSGSESGTDFTLTISSLQPEDFATYYCQQHYDTPPTFGQGTKVEIKR

SEQ ID NO: 14 - protein, 108 aa, Artificial Sequence; identical to SEQ ID NO: 13
  light variable chain of modified trastuzumab scFv

SEQ ID NO: 15 - protein, 108 aa, Artificial Sequence
  light variable chain of modified trastuzumab scFv
DIQMTQSPSSLSASVGDRVTITCRASQDVLRAVAWYQQKPGKAPKLLIYLASFLYSGVPSRFSGSRSGTDFTLTISSLQPEDFATYYCQQFYTTPPTFGQGTKVEIKR

SEQ ID NO: 16 - protein, 108 aa, Artificial Sequence
  light variable chain of modified trastuzumab scFv
DIQMTQSPSSLSASVGDRVTITCRASQKVGVAVAWYQQKPGKAPKLLIYWAEFRYDGVPSRFSGSRSGTDFTLTISSLQPEDFATYYCQQFYDTPPTFGQGTKVEIKR

SEQ ID NO: 17 - protein, 108 aa, Artificial Sequence
  light variable chain of modified trastuzumab scFv
DIQMTQSPSSLSASVGDRVTITCRASQKVGTAVAWYQQKPGKAPKLLIYSAEFLYSGVPSRFSGSRSGTDFTLTISSLQPEDFATYYCQQHYDTPPTFGQGTKVEIKR

SEQ ID NO: 18 - protein, 108 aa, Artificial Sequence
  light variable chain of modified trastuzumab scFv
DIQMTQSPSSLSASVGDRVTITCRASQKVGTAVAWYQQKPGKAPKLLIYDAEFLYSGVPSRFSGSRSGTDFTLTISSLQPEDFATYYCQQHYDTPPTFGQGTKVEIKR

SEQ ID NO: 19 - protein, 108 aa, Artificial Sequence; identical to SEQ ID NO: 3
  light variable chain of modified trastuzumab scFv

SEQ ID NO: 20 - protein, 58 aa, Artificial Sequence
  Synthetic sequence
AMNITCTGRGPDNCIQCAHYIDGPHCVKTCPAGVMGENNTLVWKYADAGHVCHLCHPN

SEQ ID NO: 21 - protein, 59 aa, Artificial Sequence
  Synthetic sequence
NGSVTCFGPEADQCVACAHYKDPPFCVARCPSGVKPDLSYMPIWKFPDEEGACQPCPIN

SEQ ID NO: 22 - protein, 57 aa, Artificial Sequence
  Synthetic sequence
EGTATCNGSGSDTCAQCAHFRDGPHCVSSCPHGVLGAKGPIYKYPDVQNECRPCHEN

SEQ ID NO: 23 - protein, 119 aa, Artificial Sequence
  modified trastuzumab scFv
EVQLVESGGGLVQPGGSLRLSCAASGFNIKDTYIHWVRQAPGKGLEWVAKIYPTEGDTKYADSVKGRFTISADTSKNTAYLQMNSLRAEDTAVYYCSRWGGDAKYAMDYWGQGTLVTVS

SEQ ID NO: 24 - protein, 119 aa, Artificial Sequence
  heavy variable chain of modified trastuzumab scFv
EVQLVESGGGLVQPGGSLRLSCAASGFNIKDTYIHWVRQAPGKGLEWVARIYPTNGDTRYADSVKGRFTISADTSKNTAYLQMNSLRAEDTAVYYCSRWGGDGKYAMDYWGQGTLVTVS

SEQ ID NO: 25 - protein, 119 aa, Artificial Sequence
  heavy variable chain of modified trastuzumab scFv
EVQLVESGGGLVQPGGSLRLSCAASGFNIKDTYIHWVRQAPGKGLEWVARIYPTNGDTKYADSVKGRFTISADTSKNTAYLQMNSLRAEDTAVYYCSRWGGDGKYAMDYWGQGTLVTVS

SEQ ID NO: 26 - protein, 119 aa, Artificial Sequence
  heavy variable chain of modified trastuzumab scFv
EVQLVESGGGLVQPGGSLRLSCAASGFNIKDTYIHWVRQAPGKGLEWVAKIYPTNGHTKYADSVKGRFTISADTSKNTAYLQMNSLRAEDTAVYYCSRWGGDGFYAMDYWGQGTLVTVS

SEQ ID NO: 27 - protein, 119 aa, Artificial Sequence
  heavy variable chain of modified trastuzumab scFv
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVS

SEQ ID NO: 28 - protein, 119 aa, Artificial Sequence
  heavy variable chain of modified trastuzumab scFv
EVQLVESGGGLVQPGGSLRLSCAASGFNIKDTYIHWVRQAPGKGLEWVARIYPTNGDTFYADSVKGRFTISADTSKNTAYLQMNSLRAEDTAVYYCSRWGGDGFYAMDYWGQGTLVTVS

SEQ ID NO: 29 - protein, 119 aa, Artificial Sequence; identical to SEQ ID NO: 24
  heavy variable chain of modified trastuzumab scFv

SEQ ID NO: 30 - protein, 119 aa, Artificial Sequence
  heavy variable chain of modified trastuzumab scFv
EVQLVESGGGLVQPGGSLRLSCAASGFNIKDTYIHWVRQAPGKGLEWVARIYPTNGDTKYADSVKGRFTISADTSKNTAYLQMNSLRAEDTAVYYCSRWGGDGFYAMDYWGQGTLVTVS

SEQ ID NO: 31 - protein, 119 aa, Artificial Sequence
  heavy variable chain of modified trastuzumab scFv
EVQLVESGGGLVQPGGSLRLSCAASGFNIKDTYIHWVRQAPGKGLEWVARIYPTNGDTKYADSVKGRFTISADTSKNTAYLQMNSLRAEDTAVYYCSRWGGVGKYAMDYWGQGTLVTVS

SEQ ID NO: 32 - protein, 119 aa, Artificial Sequence; identical to SEQ ID NO: 26
  heavy variable chain of modified trastuzumab scFv

SEQ ID NO: 33 - protein, 119 aa, Artificial Sequence; identical to SEQ ID NO: 27
  heavy variable chain of modified trastuzumab scFv

SEQ ID NO: 34 - protein, 119 aa, Artificial Sequence; identical to SEQ ID NO: 28
  heavy variable chain of modified trastuzumab scFv

SEQ ID NO: 35 - protein, 119 aa, Artificial Sequence; identical to SEQ ID NO: 27
  heavy variable chain of modified trastuzumab scFv

SEQ ID NO: 36 - protein, 119 aa, Artificial Sequence; identical to SEQ ID NO: 28
  heavy variable chain of modified trastuzumab scFv

SEQ ID NO: 37 - protein, 119 aa, Artificial Sequence; identical to SEQ ID NO: 25
  heavy variable chain of modified trastuzumab scFv

SEQ ID NO: 38 - protein, 119 aa, Artificial Sequence; identical to SEQ ID NO: 26
  heavy variable chain of modified trastuzumab scFv

SEQ ID NO: 39 - protein, 119 aa, Artificial Sequence; identical to SEQ ID NO: 27
  heavy variable chain of modified trastuzumab scFv

SEQ ID NO: 40 - protein, 119 aa, Artificial Sequence; identical to SEQ ID NO: 28
  heavy variable chain of modified trastuzumab scFv

SEQ ID NO: 41 - protein, 119 aa, Artificial Sequence
  heavy variable chain of modified trastuzumab scFv
EVQLVESGGGLVQPGGSLRLSCAASGFNIKDTYIHWVRQAPGKGLEWVARIYPTTGDTKYADSVKGRFTISADTSKNTAYLQMNSLRAEDTAVYYCSRWGGDGKYAMDYWGQGTLVTVS

(19) United States
(12) Patent Application Publication
(10) Pub. No.: US 2013/0108547 A1
(43) Pub. Date: May 2, 2013

(54) Recombinantly Produced Antibodies Targeting ErbB Signaling Molecules and Methods of Use Thereof for the Diagnosis and Treatment of Disease

(71) Applicants: Roland Dunbrack, Philadelphia, PA (US); Matthew K. Robinson, Blue Bell, PA (US); Andreas Lehmann, Bear, DE (US); Gregory P. Adams, Hatboro, PA (US)

(72) Inventors: Roland Dunbrack, Philadelphia, PA (US); Matthew K. Robinson, Blue Bell, PA (US); Andreas Lehmann, Bear, DE (US); Gregory P. Adams, Hatboro, PA (US)

(73) Assignee: FOX CHASE CANCER CENTER, Philadelphia, PA (US)

(21) Appl. No.: 13/697,653

(22) PCT Filed: May 12, 2011
(86) PCT No.: PCT/US11/36234

§ 371 Date: Dec. 21, 2012

Related U.S. Application Data
(60) Provisional application No. 61/334,334, filed on May 13, 2010.

(51) Int. Cl. C07K 16/28; A61K 39/395; A61K 45/06

(52) U.S. Cl. CPC C07K 16/2863; A61K 45/06; A61K 39/39558
USPC 424/1.49; 530/389.7; 530/388.22; 530/387.3; 530/391.3; 424/174.1; 424/183.1; 424/178.1; 424/143.1; 424/135.1; 435/320.1; 435/252.3; 435/254.2; 424/9.1

(57) ABSTRACT

Compositions and methods useful for the diagnosis and treatment of cancer are provided.

This application claims priority to U.S. Provisional Application 61/334,334 filed May 13, 2010, the entire contents being incorporated herein by reference as though set forth in full.

### Field of the Invention

This invention relates to the fields of immunology and cancer treatment. Specifically, compositions and methods for improved detection of ErbB signaling molecules (e.g., EGFR, ErbB2, ErbB3 and ErbB4) and cells expressing the same are disclosed. Also provided are methods of use of such compositions for the diagnosis and treatment of diseases associated with aberrant expression and/or function of these signaling molecules.

### BACKGROUND OF THE INVENTION

Several publications and patent documents are cited throughout the specification in order to describe the state of the art to which this invention pertains. Each of these citations is incorporated herein by reference as though set forth in full.

Antibodies have emerged as significant agents for the treatment of a number of diseases including cancer and autoimmunity. However, most of the antibodies currently used in clinical practice were developed from humanized or chimeric molecules based on mouse monoclonal antibodies. Recent advances in antibody selection and engineering techniques have led to the development of antibodies specific for highly conserved targets, the creation of novel antibody-based structures, significant improvements in affinity for target antigens, enhanced ability to engage immune effector functions, and the creation of fusion proteins with direct cytotoxic properties.

Antibodies that have potential for the greatest clinical impact are often those that directly mediate a biological effect either by initiating a signaling event (e.g., the initiation of apoptosis via anti-TRAIL antibodies) or by blocking ligand binding and inhibiting signaling (e.g., the inhibition of the binding of EGF to EGFR). However, as ligand-binding sites and other functional regions on antigens often exhibit a high degree of conservation between species, it is typically difficult or impossible to generate the desired functional antibodies using current technologies. Furthermore, the generation/isolation of antibodies specific for relevant biological targets usually results in the targeting of essentially random epitopes on the surface of the protein rather than antibodies focused on a desired functional region or epitope.

The members of the epidermal growth factor receptor (EGFR) family are overexpressed in a variety of malignancies (e.g., squamous cell carcinomas of the head and neck) and are frequently linked to aggressive disease and a poor prognosis. Although clinically effective monoclonal antibodies (MAbs) have been developed to target HER2 and EGFR, the remaining two family members, HER3 and HER4, have not been the subject of significant efforts. It is an object of the present invention to provide recombinantly produced antibodies which recognize defined epitopes on EGFR and HER3. These recombinantly engineered antibodies provide a valuable and clinically relevant panel of agents to target the members of the EGFR family for the treatment of disease.

### SUMMARY OF THE INVENTION

In accordance with the present invention, scFv antibodies that have been designed through use of molecular modeling strategies to bind specifically to predetermined epitopes on members of the EGFR family are provided. These scFvs have been designed to bind to epitopes on EGFR and HER3, which are homologous to that bound by trastuzumab on HER2.

Such antibodies include, monoclonal, polyclonal, diabodies, tribodies, single domain antibodies, and scFvs. In a particular embodiment, the antibody molecules are single-chain Fv antibody molecules. In another embodiment of the invention, the single chain Fv antibody molecules comprise an amino acid sequence selected from the group consisting of SEQ ID NOS: shown in FIG. 5.

In yet another embodiment, molecules comprising the single chain Fv antibody molecules of the invention are disclosed. Such molecules include without limitation, a diabody, a a tribody, a tetrabody, an immunotoxin, a recombinantly produced IgG, Fab, Fab′, F(ab′)₂, F(v), scFv, scFv₂, scFv-Fc, minibody, a bispecific antibody, an Affibody®, and a peptabody.

In a preferred embodiment, the antibody of the invention has binding affinity for an ErbB signaling molecule selected from the group consisting of EGFR, ErbB2, ErbB3 and ErbB4. In a particularly preferred embodiment the antibody is immunologically specific for ErbB3.

According to another aspect of the invention, compositions and methods for treating cancer are provided wherein a patient is administered a therapeutically effective amount of the anti-ErbB signaling molecules of the invention in a pharmaceutically acceptable carrier. In a particular embodiment of the invention, the cancer is selected from the group consisting of breast, squamous cell carcinoma of the head and neck (SCCHN), prostate, cervical, ovarian, testicular, and pulmonary cancers. In a specific embodiment, the cancer is breast cancer. In accordance with another aspect of the instant invention, the antibody molecule(s) can be conjugated to at least one of the following agents, a chemotherapeutic agent, a radioisotope, a toxin, a magnetic bead, a detectable label and a pro-drug activating enzyme.

In yet another embodiment of the invention, the antibodies are administered to a patient in combination with, prior to, or after administration of chemotherapeutic agents.

According to yet another aspect of the invention, compositions and methods for imaging cancer, particularly breast, SCCHN or ovarian cancer, are provided wherein a patient is administered a sufficient amount of an antibody molecule of the invention. In another embodiment, the antibody is labeled with a radioisotope and/or a contrast agent. The patient can be scanned by medical devices such as, without limitation, gamma cameras, mammography instruments, positron emission tomography (PET) cameras, and magnetic resonance imaging (MRI) imaging.

### BRIEF DESCRIPTION OF THE DRAWINGS

FIG. 1. A schematic diagram of Erb signaling is shown.

FIG. 2. Structural comparison of ErbB family members. A) Overlay of EGFR domain IV onto crystal structure HER2. EGFR is presented in cyan and HER2 in rainbow (domain IV is in orange and red). B) Crystal structure of Trastuzumab Fab fragment (purple) bound to HER2 extracellular domain (in rainbow colors) with EGFR domain IV (in cyan) overlaid to demonstrate structural similarity of epitope.

FIG. 3. Location of Trastuzumab epitope mapped onto ErbB family members. HER2 (center) residues contacted by trastuzumab are depicted as spheres in crystal structure and highlighted in primary sequence. Homologous residues in EGFR (left) and HER3 (right) are depicted similarly. Note: loop on lower right of each structure is present in the EGFR and HER3 structures but is disordered in the HER2 structure.

FIG. 4. Preliminary protein design predicted to convert specificity of trastuzumab from HER2 to HER3. Mutations depicted in the light chain are D28K, N300, S50D, S52E, R66E, T93D, H91F, Y92W. Mutations depicted in the heavy chain are Y57N, R59F. HER3=green, trastuzumab light chain in magenta and heavy chain in cyan.

FIG. 5. Mutations to the trastuzumab coding sequences which alter binding specificity are shown. In all cases the amino acids highlighted in red differ from the parent trastuzumab and are predicted to contribute to altering the specificity of the scFv. Single-chain Fv will be expressed as single polypeptide with the variable light and variable heavy sequences joined by a peptide linker, denoted as (X) and comprised of either multiple iterations of a Gly4Ser repeat or another flexible peptide.

FIG. 6. Surface plasmon resonance (SPR) analysis of SEQ3 binding to EGFR. Purified SEQ3 scFv protein exhibited a concentration dependent increase in binding to EGFR when analyzed by SPR on a BlAcore 1000. Figure depicts double subtracted data.

### DETAILED DESCRIPTION OF THE INVENTION

In accordance with the present invention, antibodies exhibiting altered specificity for ErbB signaling molecules (e.g., EGFR, ErbB2, ErbB3 and ErbB4) and methods of use thereof are provided. Specifically, methods for the immunodetection and imaging of cancer associated with aberrant ErbB signaling and expression and methods of treating the same are provided.

The antibodies of the invention include monoclonal, polyclonal, scFv and molecules comprising a plurality of scFv. Also encompassed by the present invention are conjugates of the antibody molecules described herein. Such conjugates include, without limitation, antibodies operably linked to imaging reagents, contrast agents, chemotherapeutic agents, cytotoxic molecules (e.g., immunotoxins) and the like.

I. The following definitions are provided to facilitate an understanding of the present invention.

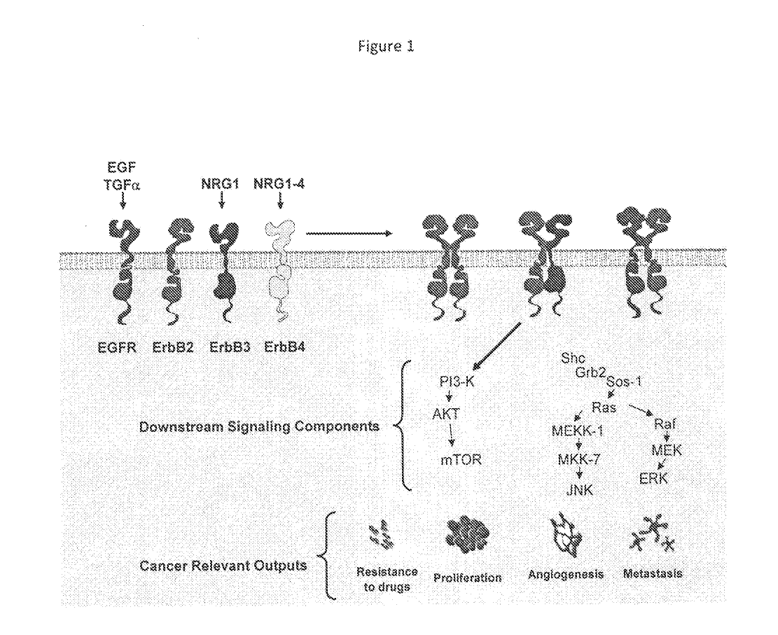

The phrase “ErbB signaling molecule” refers to a receptor selected from the group consisting of EGFR, ErbB2, ErbB3, and ErbB4. These molecules are referred to interchangeably herein and in the art as EGFR, HER2, HERS and HER4 respectively. See FIG. 1. Recombinant antibodies immunologically specific for these signaling molecules can be generated using the methodology disclosed herein. Such antibodies have utility in the diagnosis and treatment of cancer.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” may refer to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

The term “primer” as used herein refers to a DNA oligonucleotide, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos: 4,683,195, 4,800,195, and 4,965,188, the entire disclosures of which are incorporated by reference herein.

The terms “percent similarity”, “percent identity” and “percent homology”, when referring to a particular sequence are used as set forth in the University of Wisconsin GCG software program.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

The term “promoters” or “promoter” as used herein can refer to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists. A promoter from one organism can be utilized to enhance recombinant product expression from a DNA sequence that originates from another organism. For example, a vertebrate promoter may be used for the expression of jellyfish GFP in vertebrates. In addition, one promoter element can increase an amount of recombinant products expressed for multiple DNA sequences attached in tandem. Hence, one promoter element can enhance the expression of one or more recombinant products. Multiple promoter elements are well-known to persons of ordinary skill in the art.

The terms “transfected” and “transfection” as used herein refer to methods of delivering exogenous DNA into a cell. These methods involve a variety of techniques, such as treating cells with high concentrations of salt, an electric field, liposomes, polycationic micelles, or detergent, to render a host cell outer membrane or wall permeable to nucleic acid molecules of interest. These specified methods are not limiting and the invention relates to any transformation technique well known to a person of ordinary skill in the art.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide,” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The phrase “operably linked,” as used herein, may refer to a nucleic acid sequence placed into a functional relationship with another nucleic acid sequence. Examples of nucleic acid sequences that may be operably linked include, without limitation, promoters, cleavage sites, purification tags, transcription terminators, enhancers or activators and heterologous genes which when transcribed and translated will produce a functional product such as a protein, ribozyme or RNA molecule.

The phrase “solid support” refers to any solid surface including, without limitation, any chip (for example, silica-based, glass, or gold chip), glass slide, membrane, bead, solid particle (for example, agarose, sepharose, polystyrene or magnetic bead), column (or column material), test tube, or microtiter dish.

The phrases “affinity tag,” “purification tag,” and “epitope tag” may all refer to tags that can be used to effect the purification of a protein of interest. Purification/affinity/epitope tags are well known in the art (see Sambrook et al., 2001, Molecular Cloning, Cold Spring Harbor Laboratory) and include, but are not limited to: polyhistidine tags (e.g. 6×His), polyarginine tags, glutathione-S-transferase (GST), maltose binding protein (MBP), S-tag, influenza virus HA tag, thioredoxin, staphylococcal protein A tag, the FLAG™ epitope, AviTag epitope (for subsequent biotinylation), dihydrofolate reductase (DHFR), an antibody epitope (e.g., a sequence of amino acids recognized and bound by an antibody), and the c-myc epitope.

A “cell line” is a clone of a primary cell or cell population that is capable of stable growth in vitro for many generations.

An “immune response” signifies any reaction produced by an antigen, such as a viral antigen, in a host having a functioning immune system. Immune responses may be either humoral in nature, that is, involve production of immunoglobulins or antibodies, or cellular in nature, involving various types of B and T lymphocytes, dendritic cells, macrophages, antigen presenting cells and the like, or both. Immune responses may also involve the production or elaboration of various effector molecules such as cytokines, lymphokines and the like. Immune responses may be measured both in in vitro and in various cellular or animal systems. Such immune responses may be important in protecting the host from disease and may be used prophylactically and therapeutically.

An “antibody” or “antibody molecule” is any immunoglobulin, including antibodies and fragments thereof, that binds to a specific antigen. The term includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, F(v), scFv, scFv₂, scFv-Fc, minibody, diabody, tetrabody, single variable domain (e.g., variable heavy domain, variable light domain), bispecific, Affibody® molecules (Affibody, Bromma, Sweden), and peptabodies (Terskikh et al. (1997) PNAS 94:1663-1668). Dabs can be composed of a single variable light or heavy chain domain. In a certain embodiment of the invention, the variable light domain and/or variable heavy domain specific for MISIIR are inserted into the backbone of the above mentioned antibody constructs. Methods for recombinantly producing antibodies are well known in the art. For example, commercial vectors comprising constant genes to make IgGs from scFvs are provided by Lonza Biologics (Slough, United Kingdom).

“Fv” is an antibody fragment which contains an antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although often at a lower affinity than the entire binding site.

“Single-chain Fv” or “scFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv see, for example, Plückthun, A. in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) on the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Holliger et al., (1993) Proc. Natl. Acad. Sci. USA, 90: 6444-6448.

With respect to antibodies, the term “immunologically specific” refers to antibodies that bind to one or more epitopes of a protein or compound of interest, but which do not substantially recognize and bind other molecules in a sample containing a mixed population of antigenic biological molecules. As used herein, the term “immunotoxin” refers to chimeric molecules in which antibody molecules or fragments thereof are coupled or fused (i.e., expressed as a single polypeptide or fusion protein) to toxins or their subunits. Toxins to be conjugated or fused can be derived form various sources, such as plants, bacteria, animals, and humans or be synthetic toxins (drugs), and include, without limitation, saprin, ricin, abrin, ethidium bromide, diptheria toxin, Pseudomonas exotoxin, PE40, PE38, saporin, gelonin, RNAse, protein nucleic acids (PNAs), ribosome inactivating protein (RIP), type-1 or type-2, pokeweed anti-viral protein (PAP), bryodin, momordin, and bouganin.

The term “conjugated” refers to the joining by covalent or noncovalent means of two compounds or agents of the invention.

Chemotherapeutic agents are compounds that exhibit anticancer activity and/or are detrimental to a cell (e.g., a toxin). Suitable chemotherapeutic agents include, but are not limited to: toxins (e.g., saporin, ricin, abrin, ethidium bromide, diptheria toxin, Pseudomonas exotoxin, and others listed above; thereby generating an immunotoxin when conjugated or fused to an antibody); alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). Preferably, the chemotheraputic agent is selected from the group consisting of placitaxel (Taxol®), cisplatin, docetaxol, carboplatin, vincristine, vinblastine, methotrexate, cyclophosphamide, CPT-1 1, 5-fluorouracil (5-FU), gemcitabine, estramustine, carmustine, adriamycin (doxorubicin), etoposide, arsenic trioxide, irinotecan, and epothilone derivatives. Such agents can also include maytansanoids and auristatins.

The term “pro-drug” refers to a compound which is transformed in vivo to an active form of the drug. The pro-drug may be transformed to an active form only upon reaching the target in vivo or upon internalization by the target cell.

Radioisotopes of the instant invention include, without limitation, positron-emitting isotopes and alpha-, beta-, gamma-, Auger- and low energy electron-emitters. The radioisotopes include, without limitation: ¹³N, ¹⁸F, ₃₂P, ⁶⁴Cu, ⁶⁶Ga, ⁶⁷Ga, ⁶⁸Ga, ⁶⁷Cu, ⁷⁷Br, ^{80m}Br, ⁸²Rb, ⁸⁶Y, ⁸⁹Zr, ⁹⁰Y, ⁹⁵Ru, ⁹⁷Ru, ^{99m}Tc, ¹⁰³Ru, ¹⁰⁵Ru, ¹¹¹In, ^{113m}In, ¹¹³Sn, ^{121m}Te, ^{122m}Te, ^{125m}Te, ¹²³I, ¹²⁴I, ¹²⁵I, ¹²⁶I, ¹³¹I, ¹³³I, ¹⁶⁵Tm, ¹⁶⁷Tm, ¹⁶⁸Tm, ¹⁷⁷Lu, ¹⁸⁶Re, ¹⁸⁸Re, ^{195m}Hg, ²¹¹At, ²¹²Bi, ²¹³Bi, ²²⁵Ac. When the conjugated antibodies of the instant invention are employed for radio-immunodetection, the radioisotope is preferably a gamma-emitting isotope. When the conjugated antibodies of the instant invention are employed for detection by ImmunoPET (positron emission tomography), the radioisotope is preferably a positron-emitting isotope such as, without limitation, ¹³N, ¹⁸F, ⁸²Rb. When the conjugated antibodies of the instant invention are employed for radioimmunotherapy (i.e., the treating of a patient with cancer), the radioisotope is preferably selected from the group consisting of ⁸⁹Zr, ⁹⁰Y, ¹³¹I, ¹⁷⁷Lu, and ¹⁸⁶Re, although other radionuclides such as many of those listed above are also suitable. When the conjugated antibodies of the instant invention are employed for radioimmunoimaging (i.e., the diagnosis, staging, or monitoring of therapeutic response of a patient with cancer), the radioisotope is preferably selected from the group consisting of ¹²⁴I, ¹⁸F, and ¹¹¹In.

The term “radiosensitizer”, as used herein, is defined as a molecule administered to animals in therapeutically effective amounts to increase the sensitivity of the cells to radiation. Radiosensitizers are known to increase the sensitivity of cancerous cells to the toxic effects of radiation. Radiosensitizers include, without limitation, 2-nitroimidazole compounds, and benzotriazine dioxide compounds, halogenated pyrimidines, metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), 5-iododeoxyuridine (IUdR), bromodeoxycytidine, fluorodeoxyuridine (FudR), hydroxyurea, cisplatin, and therapeutically effective analogs and derivatives of the same.

### II. Preparation of Antibody Molecules

The antibody molecules of the invention may be prepared using a variety of methods known in the art. Polyclonal and monoclonal antibodies are prepared as described in Current Protocols in Molecular Biology, Ausubel et al. eds. Antibodies may be prepared by chemical cross-linking, hybrid hybridoma techniques and by expression of recombinant antibody fragments expressed in host cells, such as bacteria or yeast cells.

In one embodiment of the invention, the antibody molecules are produced by expression of recombinant antibody fragments in host cells. The genes for several of the antibody molecules that target ErbB receptors have been cloned. The nucleic acid molecules encoding the anti-ErbB antibody fragments are inserted into expression vectors and introduced into host cells. The resulting antibody molecules are then isolated and purified from the expression system. The antibodies optionally comprise a purification tag by which the antibody can be purified.

The purity of the antibody molecules of the invention may be assessed using standard methods known to those of skill in the art, including, but not limited to, ELISA, immunohistochemistry, ion-exchange chromatography, affinity chromatography, immobilized metal affinity chromatography (IMAC), size exclusion chromatography, polyacrylamide gel electrophoresis (PAGE), western blotting, surface plasmon resonance and mass spectroscopy.

### III. Uses of Anti-ErbB Receptor Antibody Molecules

Anti-ErbB antibodies (e.g., those immunologically specific for EGFR, ErbB2, ErbB3 and/or ErbB4) have broad applications in therapy and diagnosis. Specifically, such antibodies may be used: (1) to directly alter the growth of tumors that express one or more ErbB receptors; (2) to alter the growth of tumors that express ErbB receptors in combination with other cytotoxic agents; (3) to image tumors that express one or more ErbB receptors; and (4) as a diagnostic tool.

1) The antibody molecules of the instant invention can be administered to a patient in need thereof, as described hereinbelow. The antibody molecules of the instant invention include the antibodies alone and antibodies conjugated to other agents such as, without limitation, chemotherapeutic agents, radioisotopes, pro-drugs, pro-drug activating enzymes capable of converting a pro-drug to its active form, and magnetic beads (see, for example, U.S. Pat. No. 6,645,731). If the compound to be conjugated is proteinaceous, a fusion protein may be generated with the antibody molecule. Radiosensitizers may also be administered with the antibodies.

2) To alter the growth of tumors that express one or more ErbB receptors, the antibody molecules of the instant invention may be administered to a patient in combination with other cytotoxic agents. These other cytotoxic agents include, without limitation, chemotherapeutic agents, external beam radiation, targeted radioisotopes, and other antibodies or signal transduction inhibitors. Radiosensitizers may also be administered with the antibodies.

3) When employed for imaging tumors, the recombinant antibody molecules of the invention can be conjugated to radioisotopes as described hereinabove. The antibody molecules can be conjugated to the radioisotopes by any method including direct conjugation and by linking through a chelator (see, for example, U.S. Pat. No. 4,624,846). The antibody molecules may also be conjugated to labels or contrast agents such as, without limitation, paramagnetic or superparamagnetic ions for detection by MRI imaging and optical and fluorescence and/or mammography agents (examples of other labels are provided in, for example, U.S. Pat. Nos. 3,817,837; 3,850,752; 3,939,350; 3,996,345; 4,277,437; 4,275,149 and 4,366,241). Paramagnetic ions include, without limitation, Gd(III), Eu(III), Dy(III), Pr(III), Pa(IV), Mn(II), Cr(III), Co(III), Fe(III), Cu(II), Ni(II), Ti(III), and V(IV). Fluorescent agents include, without limitation, fluorescein and rhodamine and their derivatives. Optical agents include, without limitation, derivatives of phorphyrins, anthraquinones, anthrapyrazoles, perylenequinones, xanthenes, cyanines, acridines, phenoxazines and phenothiazines. Mammography agents include, without limitation, derivatives of iodine or metals such as gold, gold particles or gold nanoparticles.

In an alternative method, a secondary binding ligand, such as a second antibody or a biotin/avidin ligand binding arrangement, which can recognize the anti-ErbB receptor antibodies of the instant invention, may be conjugated with the agents described above instead of with the anti-ErbB antibody molecules. The conjugated secondary binding ligand can then be used in conjunction with anti-ErbB antibody molecules in any of the assays described herein.

4) The antibody molecules of the invention may be used to 1) diagnose cancer in patient, 2) determine the prognosis of a patient, including stage and grade (particularly whether it is metastatic) of a tumor and its potential sensitivity to therapy, 3) determine the origin of a tumor, 4) determine the efficacy of a treatment of a patient. In one embodiment the antibody molecules are utilized to detect the presence of one or more ErbB receptors in a biological sample from a patient. The biological sample may include biopsies of various tissues including, without limitation: breast, prostate, cervical, ovarian, testicular, and pulmonary. Cellular examples of biological samples include tumor cells, blood cells, ovarian cells, prostate cells, breast cells, testicular cells, cervical cells, and lung cells. The biological sample may also be a biological fluid, wherein shed ErbB receptors can be detected, such as, without limitation, blood, serum, nipple aspirate and urine. Many immunological assays are well known in the art for assaying of biological samples for the presence of a certain protein including, without limitation: immunoprecipitations, radioimmunoassays, enzyme-linked immunosorbent assays (ELISA), immunohistochemical assays, Western blot and the like.

The presence of ErbB receptors in fluids or sites not near the tumor may be indicative of metastases. Additionally, the imaging techniques described hereinabove may be employed to monitor the size of the tumor to determine the efficacy of a treatment. In a particular embodiment of the invention, other cancer diagnostic assays can be performed to confirm the results obtained with the instant invention.

The anti-ErbB receptor antibody molecules of the invention may also be used in gene therapy for direct targeting of vehicles (liposomes, viruses etc.) containing genes to specific tumors expressing ErbB receptors. In an exemplary embodiment, liposomes may be studded by the antibody molecules of the invention to facilitate tumor specific targeting. In another embodiment, antibodies may be expressed directly on the surface of viruses or as fusions with viral coat proteins to facilitate tumor specific targeting. The genes targeted in this manner can have a direct anti-tumor effect, sensitize the tumor to other agents or increase the susceptibility of the tumor to a host immune response. Anti-cancer agents such as chemotherapeutic agents, toxins, antibodies, antisense molecules, RNAi and/or radioisotopes may also be encapsulated in liposomes so modified.

In another embodiment, the antibody molecules may be used to direct gene therapy vectors, including but not limited to modified viruses, to cells that express ErbB receptors. Viruses and other vectors may also be utilized to deliver the genes for the antibody molecules to tumor cells where they could be produced and secreted into the cellular microenvironment or, through the addition of additional intracellular targeting sequences, they could be turned into intrabodies that localize to specific cellular compartments and knockout the expression of their targets.

In yet another embodiment of the instant invention, the recombinant antibody molecules of the instant invention can be conjugated or covalently attached to another targeting agent to increase the specificity of the tumor targeting. Targeting agents can include, without limitation, antibodies, cytokines, and receptor ligands. In a particular embodiment, the targeting agent is overexpressed on the tumor as compared to normal tissue. Additionally, the antibody molecules of the instant invention can be conjugated or covalently attached to compounds which elicit an immune response such as, without limitation, cytokines.

The present invention further encompasses kits for use in detecting the expression of ErbB receptors in biological samples. Such kits may comprise the antibody molecules of the invention as well as buffers and other compositions and instruction material to be used for the detection of the ErbB receptors such as EGFR, ErbB2, ErbB3 and ErbB4.

### IV. Administration of Antibodies

The antibodies as described herein will generally be administered to a patient as a pharmaceutical preparation. The term “patient” as used herein refers to human or animal subjects. These antibodies may be employed therapeutically, under the guidance of a physician for the treatment of malignant tumors and metastatic disease.

The pharmaceutical preparation comprising the antibody molecules of the invention may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of antibody molecules in the chosen medium will depend on the hydrophobic or hydrophilic nature of the medium, as well as the size and other properties of the antibody molecules. Solubility limits may be easily determined by one skilled in the art.

As used herein, “biologically acceptable medium” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the antibody molecules to be administered, its use in the pharmaceutical preparation is contemplated.

The dose and dosage regimen of an antibody according to the invention that is suitable for administration to a particular patient may be determined by a physician considering the patient's age, sex, weight, general medical condition, and the specific condition and severity thereof for which the antibody is being administered. The physician may also consider the route of administration of the antibody, the pharmaceutical carrier with which the antibody may be combined, and the antibody's biological activity.

Selection of a suitable pharmaceutical preparation depends upon the method of administration chosen. For example, the antibodies of the invention may be administered by direct injection into any cancerous tissue or into the surrounding area. In this instance, a pharmaceutical preparation comprises the antibody molecules dispersed in a medium that is compatible with the cancerous tissue.

Antibodies may also be administered parenterally by intravenous injection into the blood stream, or by subcutaneous, intramuscular or intraperitoneal injection. Pharmaceutical preparations for parenteral injection are known in the art. If parenteral injection is selected as a method for administering the antibodies, steps must be taken to ensure that sufficient amounts of the molecules reach their target cells to exert a biological effect. The lipophilicity of the antibodies, or the pharmaceutical preparation in which they are delivered, may have to be increased so that the molecules can arrive at their target locations. Furthermore, the antibodies may have to be delivered in a cell-targeting carrier so that sufficient numbers of molecules will reach the target cells. Methods for increasing the lipophilicity of a molecule are known in the art. If a small form of the antibody is to be administered, including but not limited to a Fab fragment, a Dab, an scFv or a diabody, it may be conjugated to a second molecule such as, but not limited to polyethylene glycol (PEG) or an albumin-binding antibody or peptide to prolong its retention in blood.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral or parenteral. In preparing the antibody in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric-coated by standard techniques.

For parenterals, the carrier will usually comprise sterile water, though other ingredients, for example, to aid solubility or for preservative purposes, may be included. Injectable suspensions may also be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of antibody molecules may be determined by evaluating the toxicity of the antibody molecules in animal models. Various concentrations of antibody pharmaceutical preparations may be administered to mice with transplanted human tumors, and the minimal and maximal dosages may be determined based on the results of significant reduction of tumor size and side effects as a result of the treatment. Appropriate dosage unit may also be determined by assessing the efficacy of the antibody molecule treatment in combination with other standard anti-cancer drugs. The dosage units of antibody molecules may be determined individually or in combination with each anti-cancer treatment according to greater shrinkage and/or reduced growth rate of tumors.

The pharmaceutical preparation comprising the antibody molecules may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The following examples provide illustrative methods of practicing the instant invention, and are not intended to limit the scope of the invention in any way.

### EXAMPLE I

Rationally Designed Recombinantly Produced Antibodies with Altered Immunospecificity for ErbB Signaling Molecules

Using computational methods to improve the affinity and/or specificity of an antibody for its antigen requires a three-dimensional structure of the antigen/antibody complex followed by application of protein design computations that suggest mutations of the binding site on the antibody that will bind more tightly to the antigen. Protein design methods usually begin with the structure of a protein backbone and then sample different amino acids at each site until a sequence is determined that is likely to fold into the input backbone structure^{1,2,3,4,5}. At each step, the conformations of side chains in the protein need to be accurately predicted in order to assess whether the mutation can fit into the structure and whether it makes favorable interactions with its surroundings including a target ligand, if present. Nearly all published protein design methods use the backbone-dependent rotamer library^{6,7,8} developed by one of the present inventors in order to sample and evaluate the internal energy of different conformations (“rotamers”) of protein side chains. In recent years, such methods have been applied to binding of proteins to other molecules⁹. In this case, one molecule is the desired target and is left fixed in structure and sequence. The other is then subject to design at sites that have contact with the desired target. For antibodies, the antigen is known, and given an antigen-antibody structure (predicted or experimental), design simulations can be applied to CDR residues to improve affinity of the antibody for its antigen¹⁰.

In the absence of an experimentally determined structure of the antibody/antigen complex, a predicted structure can be used. This involves: (1) prediction of the antibody structure given its sequence; (2) computational docking of the predicted antibody structure onto the structure of the antigen, preferably with constraints derived from experimental information on where the epitope is located on the antigen. Since it was first recognized that proteins can share similar structures¹¹, computational methods have been developed to build models of proteins of unknown structure based on structures of related proteins¹². These efforts, referred to as homology or comparative modeling, follow a basic protocol: 1) for a target sequence of unknown structure, identify a template structure with sequence related to the target and align the target sequence to the template sequence and structure; 2) for core secondary structures and all well-conserved parts of the alignment, borrow the backbone coordinates of the template according to the sequence alignment with the target; 3) build side chains onto the backbone model according to the target sequence; 4) for segments of the target sequence for which coordinates cannot be borrowed from the template because of insertions and deletions in the alignment (usually in loop regions of the protein) or because of missing coordinates in the template, rebuild these regions using loop modeling methods or other ab initio structure prediction methods; 5) refine the structure, modeling likely differences in the relative positions of a helices, β sheet strands, and other elements of structure.

Antibodies present some special opportunities for more accurate structure modeling than do proteins in general. The first advantage is the near constant immunoglobulin fold. If a novel antibody sequence can be aligned to a database of antibodies of known structure, this simplifies the modeling problem for most of the structure. The other advantage is that there are ˜400 experimental structures of antibodies with different CDR or antigen-binding loop sequences imposed on closely related frameworks. Thus, CDRs of an antibody of unknown structure can be matched to similar sequences in the known structures, and the loop can be modeled using the conformation in the known structure. Fortunately, there seems to be a limited set of conformations from which most of the different CDR loop structures are drawn.

These are the canonical CDR loop classes defined by Chothia, Thornton, and others^{13,14,15,16,17,18,19.} As the overall database of antibody structures has grown, the number of observed classes of CDR loop conformations has also grown. This is particularly true of the most variable of the CDRs, H3 (the C-terminal V_{H} CDR)¹⁹. Loops that cannot be modeled based on a known structure can be modeled using ab initio structure prediction methods, such as Loopy²⁰ or Rosetta²¹.

Many methods have been developed for computational docking of two proteins^{22,23,24,25,26,27.} These methods examine many orientations and contact surfaces on the proteins, perform optimization procedures on the structure, and use a scoring function to select the most likely structure. The first criterion is usually shape complementarity, and this can be used to rule out many conformations²⁸. The second criterion is then complementarity of various characteristics of the surfaces in contact. There should be some interactions of oppositely charged residues and no strong repulsion of same-charged residues at the interface. Hydrophobic residues on one surface should be in contact with hydrophobic residues on the other surface. The best situation is when the sites on each protein are known via some experimental constraints²⁹. In this case, the proteins can be docked manually initially, and the docking programs can be used to rotate one protein on an axis connecting the two protein centers. One protein can also be moved or rotated in small increments from its initial position to sample possible scenarios of binding. On the antibody, the binding site is clearly defined, although not all antibody-antigen interactions use all six CDR loops. The binding site on the antigen, or the epitope, can be determined experimentally using a number of methods, including crosslinking³⁰, hydrogen-deuterium exchange^{31,32}, hydroxyl radical footprinting³³, and protease digestion protection^{34,35}.

Growth Factor Receptors as Targets for Rationally Designed mAb-based Cancer Therapies

Growth factor receptors, such as the ErbB family of receptor tyrosine kinases (RTKs) represent a well-established class of targets for therapeutic intervention via mAbs. Normal signaling through this family of RTKs (EGFR, HER2, HER3, and HER4; See FIG. 1) often leads to mitogenic and pro-survival responses. Unregulated signaling through EGFR and HER2, as seen in a number of common cancers due to receptor overexpression, has long been known to promote tumor cell growth and insensitivity to chemotherapeutic agents. Clinically relevant anti-EGFR (cetuximab and panitumumab) and anti-HER2 (trastuzumab and pertuzumab) mAbs block signaling through their target receptors via a variety of mechanisms. These include: a) blocking ligand binding, b) preventing dimerization, and c) altering receptor trafficking. Despite significant efforts in both the pharmaceutical and academic settings little work has been done to directly test how targeting each of these mechanisms affects signaling through individual family members and which provides the optimal therapeutic benefit. Recent literature has also identified previously unappreciated roles of HER3 in both disease progression and drug resistance.

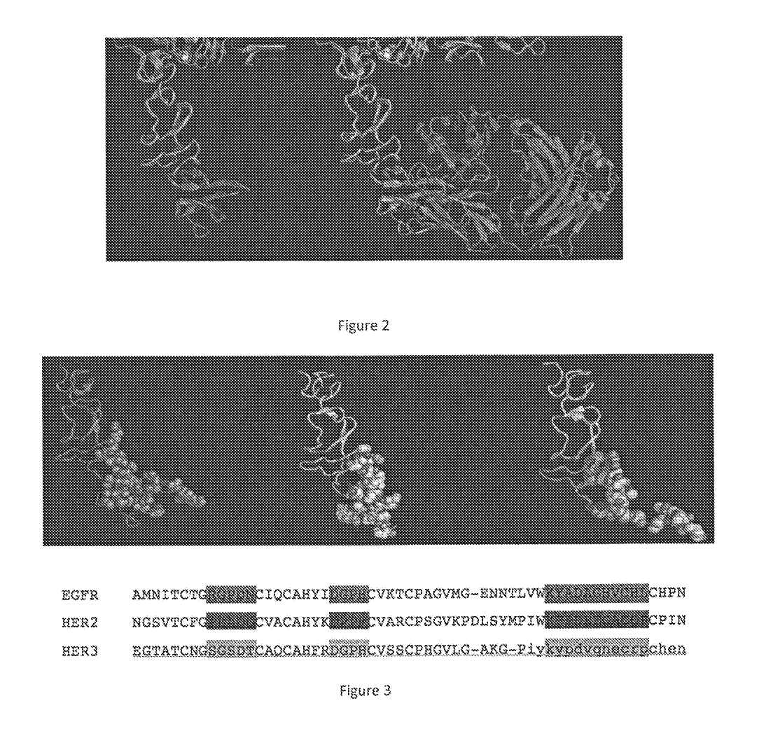

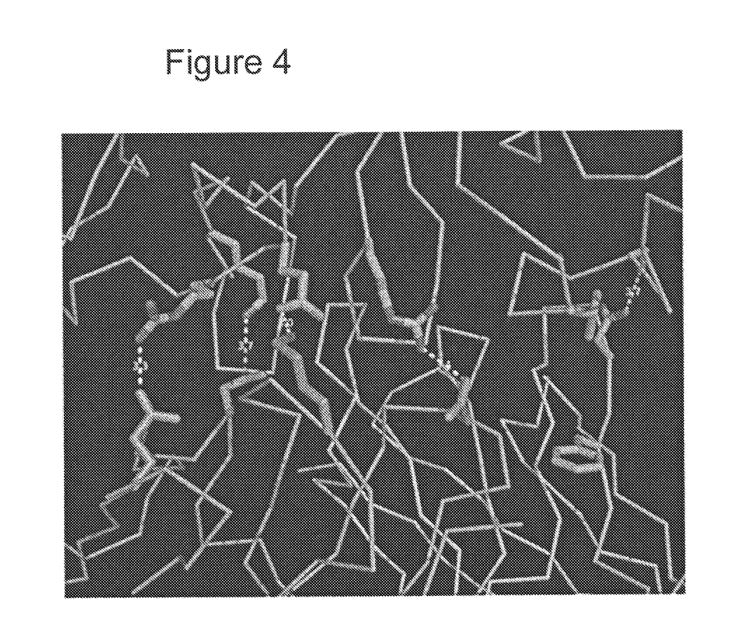

In one aspect of the invention, improved HER3 targeting molecules which have been rationally engineered to interrogate each of the above mechanisms of action are provided. Such antibodies should help elucidate the most effective mechanism for inhibiting HER3-dependent signaling. The ErbB family members display a high degree of structural conservation as depicted for domain IV in FIG. 2, but also throughout domains I-III (data not shown). The crystal structures for EGFR and HER3 used for the experiments described herein have been disclosed. See Li et al., (2005) Cancer Cell 7:301-311 for EGFR (PDB entry 1YY9); and Cho et al. (2002) Science 297:1330-1333 for HER3 (PDB entry 1M6B). Despite this structural conservation, the regions differ in primary amino acid sequences (FIG. 3). These differences presumably underlie the specificity of the clinical antibodies and will serve as the basis for our modeling to engineer mAbs that bind homologous epitopes on different ErbB family members. Based on publicly available crystal structure data of the ErbB family members, and on HER2 complexed with trastuzumab, we have superimposed domain IV of EGFR and HER3 onto domain IV of HER2 to identify homologous epitopes (FIGS. 2 and 3). With these results in hand, we have now begun computational experiments to design a modified trastuzumab that will bind either to domain IV of EGFR or to domain IV of HER3. Preliminary design results using Statistical, Computationally Assisted Design Strategy (SCADS)³⁶ that focused on CDR3 of the trastuzumab heavy chain (CDR H3) has identified an F 104K mutation in trastuzumab, which would add a salt bridge to EGFR E578, as a potential mutation for imparting different specificity (FIG. 4). Additional work will be carried out to refine this preliminary H3 design and to investigate the contribution of the additional 5 CDRs. Designs will then be engineered into trastuzumab for testing. Pertuzumab and cetuximab variants will also be developed.

Advances in antibody-engineering techniques have led to vast improvements in the efficacy of mAb-based therapies. Toward this goal, we have identified a distinct antigen system for leveraging our protein design strategies and have generated promising results. In one approach, we will use directed evolution of clinically relevant anti-EGFR and anti-HER2 mAbs to build anti-HER3 mAbs with defined mechanisms-of-action. Doing so will provide a novel and rapid method to interrogate how best to inhibit HER3-dependent signaling in different cancer settings. This target is of significant clinical importance and the biological agents we are engineering have the potential for clinical translation. The development of protein design tools necessary to precisely engineer new mAb therapeutics for indications ranging from cancer to infectious disease are highly desirable. One of the present inventors has used recently developed clustering algorithms⁴⁴ to group CDR loop structures using a novel application of a similarity criterion used in directional statistics⁴⁵. This new clustering, while similar to previous efforts by Chothia and others, significantly extends their work after more than 10 years because we used all the antibody structures available in the PDB today, and because we have used more sophisticated statistical methods than used previously. We have determined specific residues in CDRs that determine which canonical loop structure in our clustering is most likely for each CDR, so that given a target sequence for a new antibody, we can rapidly predict suitable template loop structures in the PDB. Second, we can build a model for the backbone of each CDR by grafting loops from different known structures onto a suitable backbone (a high-resolution structure, closest to the target) using our cyclic coordinate descent method. See Canutescu, A. A. and Dunbrack, R. L., Jr. Cyclic coordinate descent: A robotics algorithm for protein loop closure. Protein Sci. 12, 963-972 (2003) for closing loops. The side chains of the model will be placed with our program SCWRL4 as described in Proteins 77, 778-795 (2009). SCWRL is the most commonly used program for side-chain prediction, and is in general faster and more accurate than other programs⁴⁷. SQWRL4 is based on a faster and more robust graph theory algorithm based on tree decomposition, compared to the biconnected-component decomposition used in SCWRL3⁴⁶. It achieves higher structure prediction accuracy by allowing flexibility of the side-chain conformations about their standard conformations, and with a novel anisotropic hydrogen bonding potential. We are now adding a feature used to predict which side chains sample multiple conformations. Such information is useful in both docking and design (described below).

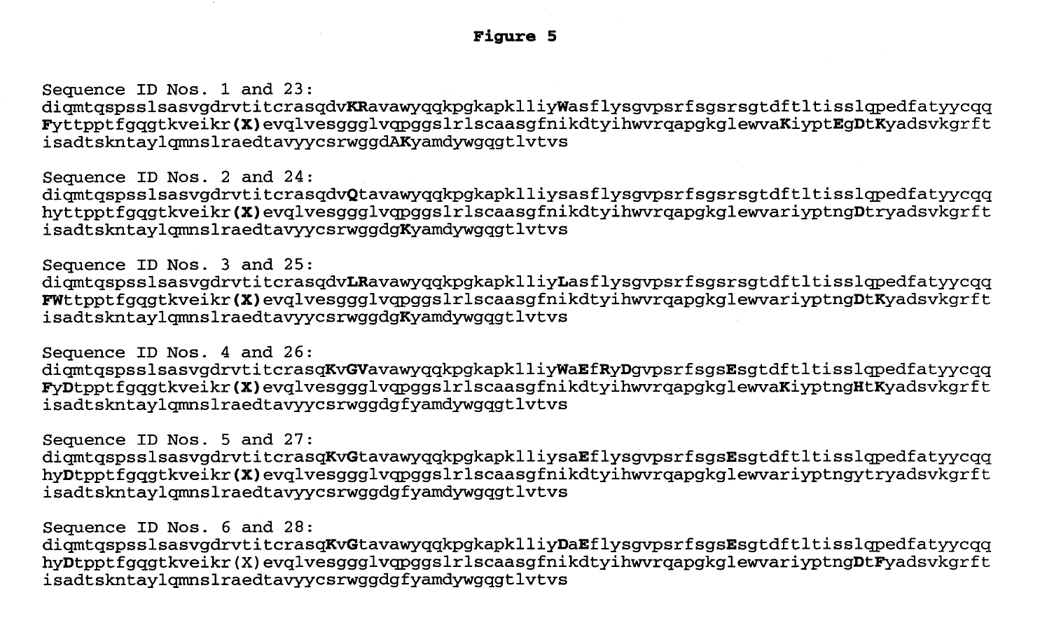

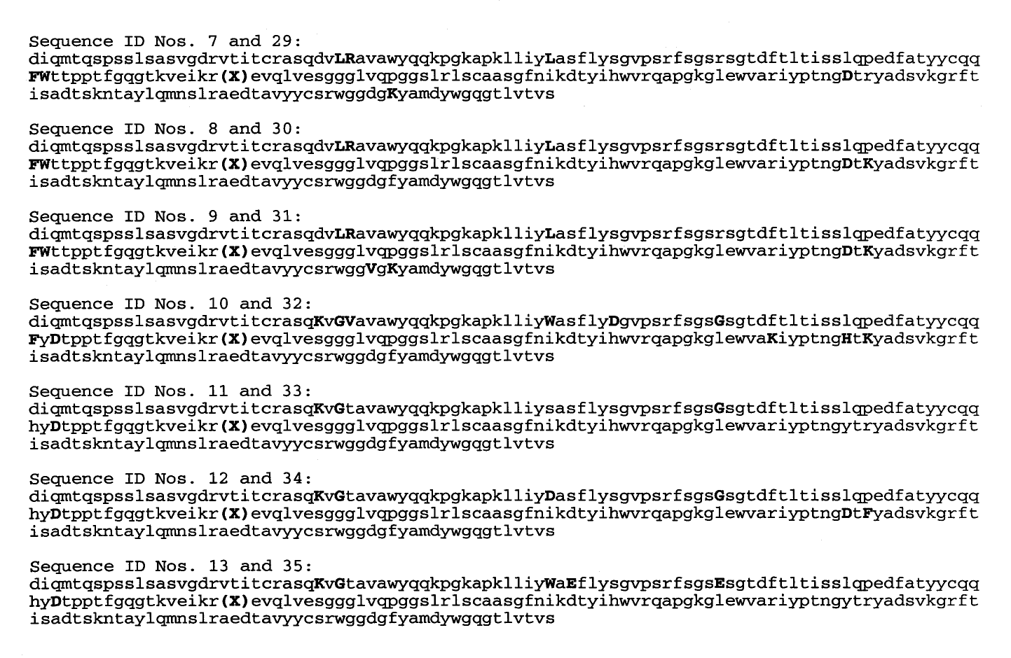

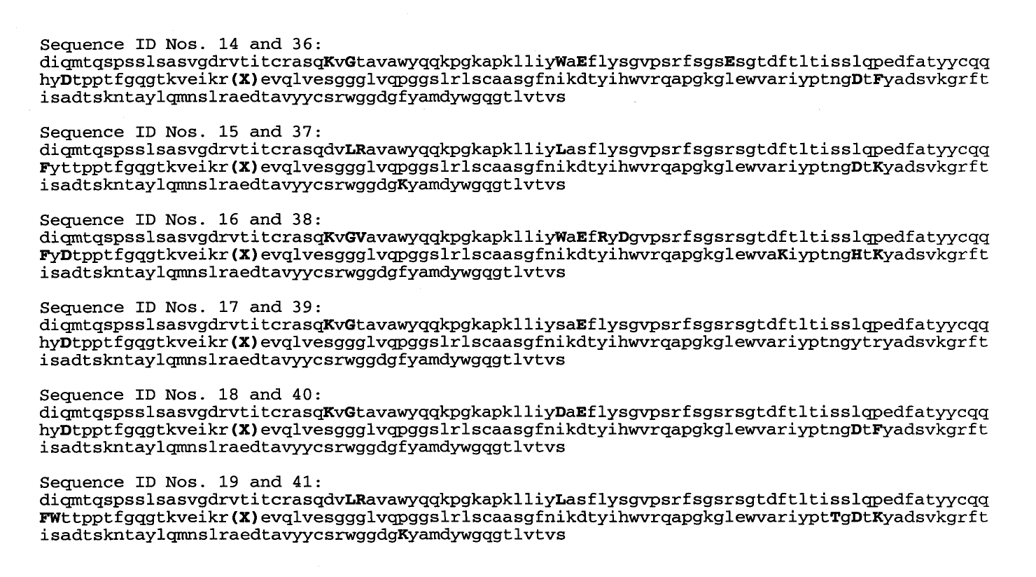

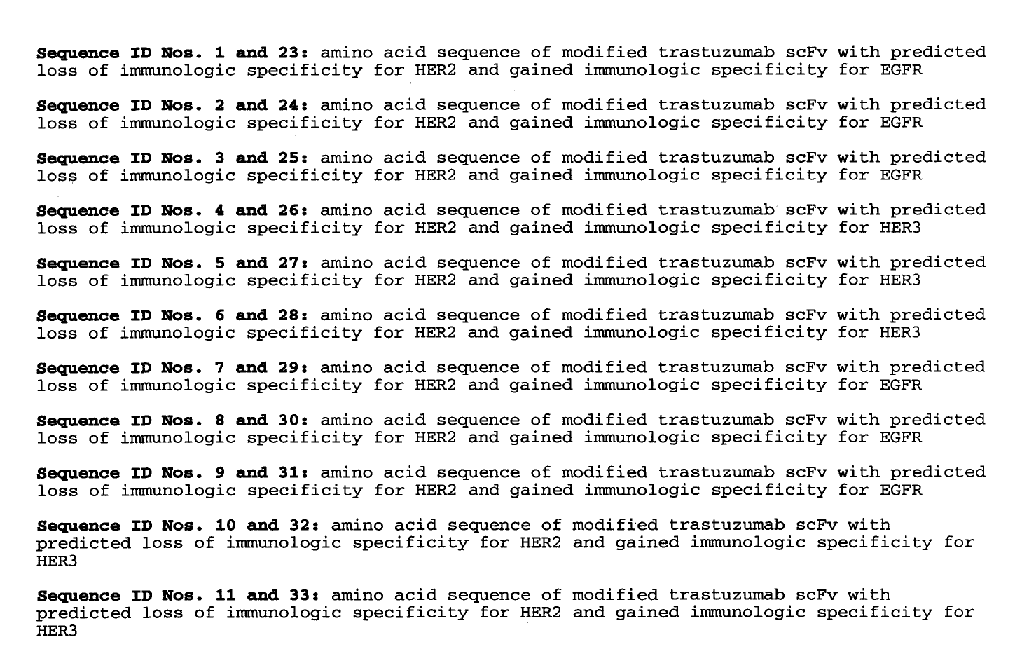

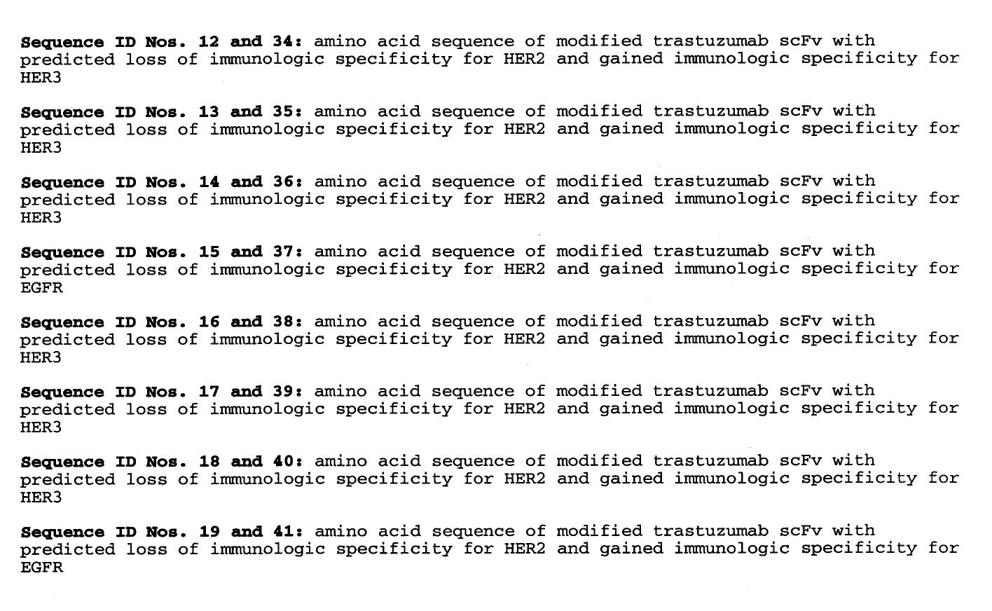

(B) Modeling antigen-antibody complexes In the HER3 or EGFR/trastuzumab project, we already have an experimental structure of HER2 bound to the antibody (PDB entry 1N8Z)⁴⁹. We have used the FATCAT program⁵° to superimpose domain IV of HER3 and EGFR onto domain IV in HER2 in complex with trastuzumab. See FIG. 5. These models will be refined using the RosettaDock program²⁶ , which allows small relative motions from an initial structure and can be used to make small changes in the backbone and side chains of either protein during model refinement.

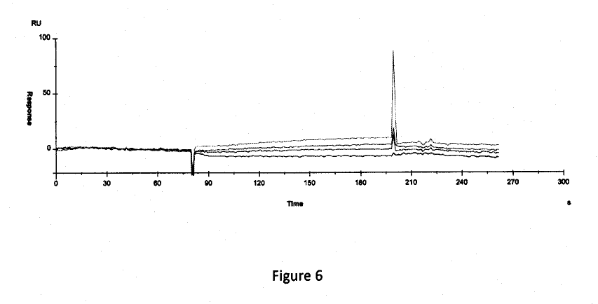

Binding of the scFv of SEQ ID NO: 3 to EGFR extracellular domains (ECDs) was characterized by SPR using EGFR ECDs (Horak et al, 2005) as target antigens and methods described previously . Briefly EGFR ECDs were immobilized on the surface of a CM5 sensor chip via NHS-ester chemistry and serially diluted samples of the scFV of SEQ ID NO: 3 (0 μM to 5 μM) were passed over the surface to monitor binding. Specific binding to EGFR was identified from double-subtracted data using the BIAEvaluation 3.2 software (BlAcore, Piscataway, N.J.). Binding to an activated and quenched flow cell was used as a negative control to subtract out the contribution of non-specific binding to the matrix and buffer effects were removed by subtracting against 0 nM SEQ3 scFv to generate the final sensorgrams. The data show that purified scFv protein of SEQ ID NO: 3 exhibited a concentration-dependent increase in binding to EGFR when analyzed by SPR on a BIAcore 1000. See FIG. 6 which depicts double subtracted data.

For antigen/antibody complexes with unknown structures, we will first model the antigen protein (if need be) and the antibody. Once we have a model (or a set of models if we choose different templates for one or more loops), we will use experimental information on the epitope as constraints on docking with the HEX^{51,52} and RosettaDock²⁶ programs to predict the structure of the antibody/antigen complex. HEX allows the user to set up an initial configuration of the two proteins and then to search a space within translational and rotational intervals of the starting conformation. HEX is fast and should provide a number of reasonable models. We have used it successfully to predict antigen-antibody complex structures based on protease digestion protection data^{34,35}. These docking studies will be refined and rescored with RosettaDock. RosettaDock is much more computer-intensive as it also allows side-chain and backbone conformations to change. However, this kind of adjustment of fine structure is important in refining a model and obtaining scores that can differentiate the most likely structure(s) from a large number of decoys.

(C) Protein Design Methods: We will apply three different methods for protein design: SCADS, RosettaDesign, and SCWRL4Design. SCADS³⁶ uses the backbone-dependent rotamer library and statistical methods to produce probabilities for each of the 20 amino acids at a fixed list of positions (e.g., certain positions in the CDRs to be designed). As SCADS provides probabilities for each amino acid type, we can choose very high probability mutations to make first, and then rerun the program keeping the fixed positions from earlier runs. This can be done iteratively until a list of mutations is compiled for experimental testing.

Second, we will use the RosettaDesign program^{4,53}. This program has been successful in designing new interactions of proteins. It allows some small backbone movements in both proteins, e.g. of protein loops, which may be very useful in forming favorable interactions between the proteins.⁵⁴ Third, we have already added the ability to perform protein design to a developmental version of our program SCWRL4. We will test and further develop “SCWRL4Design” by applying it to known protein folds and protein complexes. One of the standard tests for design programs is “sequence recovery”—the ability to recover a majority of the sequence by applying protein design to an experimental backbone structure. SCWRL4 is fast enough that it should be able to perform much more exhaustive searches in sequence space for improved affinity. We will then use SCWRL4Design to predict mutations that should alter the specificity of trastuzumab to create mAbs with specificity for HER3 and EGFR domain IV.

We have recently compiled a statistical analysis of the protein backbone in long loops, which includes information on how the backbone conformational dihedral angles of a residue are affected not only by the residue type but also the residue type of its neighbors⁶¹. For instance, a proline residue to the right nearly eliminates propensity to be in the alpha region of the dihedral angle space. This information can be used in protein design to evaluate proposed mutations on whether they would change the backbone conformation. Thus, some mutations might be inconsistent with the antibody loop conformations we have, and should not be used. We can apply this kind of filter after design calculations to eliminate some choices, or during the design calculation with our SCWRL4Design program. We will test both of these strategies.

D) Data Analysis: For both lines of research, protein design predictions will be engineered into scFv backbones using standard molecular biology techniques. Modified scFvs will be expressed in E. coli and purified as described⁵⁵. We have successfully converted scFvs into

IgGs which will be necessary for in vivo validation. Readouts of the biological activity associated with each of the constructs will be necessary to inform on their potential as therapeutics. We have significant experience measuring the therapeutic potential of ErbB-targeted agents.⁵⁷

ErbB signaling plays a pivotal role in cancer initiation and progression. Clearly, interruption of ErbB signaling could result in improved clinical outcomes in certain cancer patients. When bound to its specific epitope on HER2, trastuzumab disrupts signaling through HER2 in a manner that leads to a clinically relevant blockade of tumor cell growth.

The single-chain Fv (scFv) antibodies described here target the homologous epitopes on EGFR and HER3 with the goal of inducing a similar signaling blockade through their respective target receptors. The location of the epitope on domain IV is also well positioned to facilitate simultaneous co-targeting of specific receptor heterodimers using bispecific scFv (bs-scFV) molecules, analogous to those described by Robinson et al.⁵⁷ . By binding to the defined epitopes on both members of the heterodimer bs-scFv have the potential to simultaneously alter signaling through both family members.

Advances in engineering strategies have driven the success of mAb-based therapies. The initial jump from murine to chimeric, with subsequent advances to humanized and fully human antibodies, essentially eliminated the immunogenicity that was seen with early molecules. This step was critical in making mAbs viable drug candidates. Current trends in engineering are now focused on optimizing efficacy. We believe that the rational design approach we are proposing will: 1) allow engineering of mAb specific for a distinct, predefined epitope, 2) provide mechanism to impart “ligand functionality” to mAb through rational design, 3) enhance the speed from concept to preclinical testing, and 4) decrease costs associated with isolating and optimizing preclinical agent will be lowered as compared to conventional technologies.

### REFERENCES

1 Dahiyat, B. I. & Mayo, S. L. Protein design automation. Protein Science 5, 895-903 (1996).
2 Desjarlais, J. R. & Handel, T. M. De novo design of the hydrophobic cores of proteins. Protein Sci 4, 2006-2018 (1995).
3 Gordon, D. B. & Mayo, S. L. Branch-and-terminate: a combinatorial optimization algorithm for protein design. Structure Fold Des 7, 1089-1098 (1999).
4 Kuhlman, B. et al. Design of a novel globular protein fold with atomic-level accuracy. Science (New York, N.Y. 302, 1364-1368 (2003).
5 Malakauskas, S. M. & Mayo, S. L. Design, structure and stability of a hyperthermophilic protein variant. Nat Struct Biol 5, 470-475 (1998).
6 Dunbrack, R. L. Rotamer libraries in the 21st century. Curr. Opin. Struct. Biol. 12, 431-440 (2002).
7 Dunbrack, R. L. & Cohen, F. E. Bayesian statistical analysis of protein side-chain rotamer preferences. Protein Sci. 6, 1661-1681 (1997).
8 Shapovalov, M. V. & Dunbrack, R. L. Statistical and conformational analysis of the electron density of protein side chains. Proteins 66, 279-303 (2007).
9 Kortemme, T. & Baker, D. Computational design of protein-protein interactions. Curr Opin Chem Biol 8, 91-97 (2004).
10 Lippow, S. M., Wittrup, K. D. & Tidor, B. Computational design of antibody-affinity improvement beyond in vivo maturation. Nature biotechnology 25, 1171-1176 (2007).
11 Perutz, M. F., Kendrew, J. C. & Watson, H. C. Structure and function of haemoglobin. Journal of Molecular Biology 13, 669-678 (1965).
12 Browne, W. J., North, A. C. & Phillips, D. C. A possible three-dimensional structure of bovine alpha-lactalbumin based on that of hen's egg-white lysozyme. Journal of Molecular Biology 42, 65-86 (1969).
13 Chothia, C. & Lesk, A. M. Canonical structures for the hypervariable regions of immunoglobulins. Journal of Molecular Biology 196, 901-917 (1987).
14 Chothia, C. et al. Structural repertoire of the human VH segments. Journal of Molecular Biology 227, 799-817 (1992).
15 Chothia, C. et al. Conformations of immunoglobulin hypervariable regions Nature 342, 877-883 (1989).
16 Morea, V., Tramontano, A., Rustici, M., Chothia, C. & Lesk, A. M. Antibody structure, prediction and redesign. Biophys Chem 68, 9-16 (1997).
17 Morea, V., Tramontano, A., Rustici, M., Chothia, C. & Lesk, A. M. Conformations of the third hypervariable region in the VII domain of immunoglobulins. Journal of Molecular Biology 275, 269-294 (1998).
18 Martin, A. C., Cheetham, J. C. & Rees, A. R. Molecular modeling of antibody combining sites. Methods in enzymology 203, 121-153 (1991).
19 Oliva, B., Bates, P. A., Querol, E., Aviles, F. X. & Sternberg, M. J. Automated classification of antibody complementarity determining region 3 of the heavy chain (H3) loops into canonical forms and its application to protein structure prediction. J Mol Biol 279, 1193-1210 (1998).
20 Xiang, Z., Soto, C. S. & Honig, B. Evaluating conformational free energies: the colony enegy and its application to the problem of protein loop prediction. Proc. Natl. Acad Sci. USA 99, 7432-7437 (2002).
21 Rohl, C. A., Strauss, C. E., Chivian, D. & Baker, D. Modeling structurally variable regions in homologous proteins with rosetta. Proteins 55, 656-677 (2004).
22 Camacho, C. J. Modeling side-chains using molecular dynamics improve recognition of binding region in CAPRI targets. Proteins 60, 245-251 (2005).
23 Carter, P., Lesk, V. I., Islam, S. A. & Sternberg, M. J. Protein-protein docking using 3D-Dock in rounds 3, 4, and 5 of CAPRI. Proteins 60, 281-288 (2005).
24 Comeau, S. R., Vajda, S. & Camacho, C. J. Performance of the first protein docking server ClusPro in CAPRI rounds 3-5. Proteins 60, 239-244 (2005).
25 Fernandez-Recio, J., Totrov, M. & Abagyan, R. Soft protein-protein docking in internal coordinates. Protein Sci 11, 280-291 (2002).
26 Gray, J. J. et al. Protein-protein docking with simultaneous optimization of rigid-body displacement and side-chain conformations. J Mol Biol 331, 281-299 (2003).
27 Schneidman-Duhovny, D., Inbar, Y., Nussinov, R. & Wolfson, H. J. Geometry-based flexible and symmetric protein docking. Proteins 60, 224-231 (2005).
28 Norel, R., Lin, S. L., Wolfson, H. J. & Nussinov, R. Shape complementarity at protein-protein interfaces. Biopolymers 34, 933-940 (1994).
29 Clore, G. M. & Schwieters, C. D. Docking of protein-protein complexes on the basis of highly ambiguous intermolecular distance restraints derived from 1H/15N chemical shift mapping and backbone 15N-1H residual dipolar couplings using conjoined rigid body/torsion angle dynamics. J Am Chem Soc 125, 2902-2912 (2003).
30 Petrotchenko, E. V., Pedersen, L. C., Borchers, C. H., Tomer, K. B. & Negishi, M. The dimerization motif of cytosolic sulfotransferases. FEBS Lett 490, 39-43 (2001).

31 Baerga-Ortiz, A., Hughes, C. A., Mandell, J. G. & Komives, E. A. Epitope mapping of a monoclonal antibody against human thrombin by H/D-exchange mass spectrometry reveals selection of a diverse sequence in a highly conserved protein. Protein Sci 11, 1300-1308 (2002).
32 Mandell, J. G., Baerga-Ortiz, A., Falick, A. M. & Komives, E. A. Measurement of solvent accessibility at protein-protein interfaces. Methods in molecular biology (Clifton, N.J. 305, 65-80 (2005).
33 Kamal, J. K. & Chance, M. R. Modeling of protein binary complexes using structural mass spectrometry data. Protein Sci 17, 79-94 (2008).
34 Yi, J., Arthur, J. W., Dunbrack, R. L. & Skalka, A. M. An inhibitory monoclonal antibody binds at the turn of the helix-turn- helix motif in the N-terminal domain of HIV-1 integrase. J Biol Chem 275, 38739-38748. (2000).
35 Yi, J. et al. Mapping the epitope of an inhibitory monoclonal antibody to the C-terminal DNA-binding domain of HIV-1 integrase. J Biol Chem 277, 12164-12174 (2002).
36 Kono, H., Wang, W. & Saven, J. G. Combinatorial protein design strategies using computational methods. Methods Mol. Biol. 352, 3-22 (2007).
37 Bakkum-Gamez, J. N. et al. Mullerian inhibiting substance type II receptor (MISIIR): a novel, tissue-specific target expressed by gynecologic cancers. Gynecol Oncol 108, 141-148 (2008).
38 Segev, D. L. et al. Mullerian inhibiting substance regulates NFkappaB signaling and growth of mammary epithelial cells in vivo. J Biol Chem 276, 26799-26806 (2001).
39 Segev, D. L. et al. Mullerian-inhibiting substance regulates NF-kappa B signaling in the prostate in vitro and in vivo. Proc Natl Acad Sci USA 99, 239-244 (2002).
40 Masiakos, P. T. et al. Human ovarian cancer, cell lines, and primary ascites cells express the human Mullerian inhibiting substance (MIS) type II receptor, bind, and are responsive to MIS. Clin Cancer Res 5, 3488-3499 (1999).
41 Lefevre, G., Tran, D., Hoebeke, J. & Josso, N. Anti-idiotypic antibodies to a monoclonal antibody raised against anti-mullerian hormone exhibit anti-mullerian biological activity. Mol Cell Endocrinol 62, 125-133 (1989).
42 MacLaughlin, D. T. & Donahoe, P. K. Mullerian inhibiting substance: an update. Adv Exp Med Biol 511, 25-38; discussion 38-40 (2002).
43 Wang, Q., Canutescu, A. A. & Dunbrack, R. L. SCWRL and Mo1IDE: computer programs for side-chain conformation prediction and homology modeling. Nature Protocols 3, 1832-1847 (2008).
44 Frey, B. J. & Dueck, D. Clustering by passing messages between data points. Science (New York, N.Y. 315, 972-976 (2007).
45 Mardia, K. V. & Jupp, P. E. Directional Statistics. (Wiley, 2000).
46 Canutescu, A. A., Shelenkov, A. A. & Dunbrack, R. L., Jr. A graph-theory algorithm for rapid protein side-chain prediction. Protein Sci 12, 2001-2014 (2003).
47 Wallner, B. & Elofsson, A. All are not equal: a benchmark of different homology modeling programs. Protein Sci 14, 1315-1327 (2005).
48 Krivov, G. G., Shapovalov, M. V. & Dunbrack, R. L., Jr. Improved prediction of protein side-chain conformations with SCWRL4. Proteins (2009).
49 Cho, H. S. et al. Structure of the extracellular region of HER2 alone and in complex with the Herceptin Fab. Nature 421, 756-760 (2003).
50 Ye, Y. & Godzik, A. Flexible structure alignment by chaining aligned fragment pairs allowing twists. Bioinformatics 19, ii246-255, doi:10.1093/bioinformatics/btg1086 (2003).
51 Ritchie, D. W. Evaluation of protein docking predictions using Hex 3.1 in CAPRI rounds 1 and 2. Proteins 52, 98-106 (2003).
52 Ritchie, D. W. & Kemp, G. J. Protein docking using spherical polar Fourier correlations. Proteins 39, 178-194 (2000).
53 Dantas, G., Kuhlman, B., Callender, D., Wong, M. & Baker, D. A large scale test of computational protein design: folding and stability of nine completely redesigned globular proteins. J Mol Biol 332, 449-460 (2003).
54 Kortemme, T. et al. Computational redesign of protein-protein interaction specificity. Nat Struct Mol Biol 11, 371-379 (2004).
55 Robinson, M. K., Weiner, L. M. & Adams, G. P. Improving monoclonal antibodies for cancer therapy. Drug Discovery Research 61, 172-187 (2004).
56 Horak, E. et al. Isolation of scFvs to in vitro produced extracellular domains of EGFR family members. Cancer Biotherapy & Radiopharmaceuticals 20, 603-613 (2005).
57 Robinson, M. K. et al. Targeting ErbB2 and ErbB3 with a bispecific single-chain Fv enhances targeting selectivity and induces a therapeutic effect in vitro. Br. J. Cancer 99, 1415-1425 (2008).
58 Visser, J. A. et al. The Serine/Threonine Transmembrane Receptor ALK2 Mediates Mullerian Inhibiting Substance Signaling. Mol. Endocrinol. 15, 936-945, doi:10.1210/me.15.6.936 (2001).
59. Yuan Q A, Simmons H H, Robinson M K, Russeva M, Marasco W A, Adams G P. Development of engineered antibodies specific for the Mullerian inhibiting substance type II receptor: a promising candidate for targeted therapy of ovarian cancer. Mol Cancer Ther 2006; 5:2096-105.
60. Krivov, G., Shapovalov, M. V., and Dunbrack, R. L., Jr. Improved prediction of protein side-chain conformations with SCWRL4. Proteins 77, 778-795 (2009)].
61. Ting et al., Neighbor-dependent Ramachandran probability distributions of amino acids developed from a hierarchical Dirichlet process model. PLOS Comp. Biol. 6(4):e10000763 (2010)

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. An isolated, recombinantly produced antibody having binding affinity for an ErbB signaling receptor molecule, selected from the group consisting of at least one of EGFR, ErbB2, ErbB3 and ErbB4.

2. The antibody of claim 1, selected from the group consisting of a monoclonal antibody, a polyclonal antibody, a single chain Fv antibody, a diabody, a tribody, a tetrabody, a bispecific antibody, a single domain antibody, a minibody, an scFv-Fc molecule, a Fab fragment, a Fab′ fragment and a F(ab′)₂ fragment.

3. The antibody of claim 2, which is monoclonal.

4. The antibody of claim 2, which is a single chain Fv antibody.

5. A molecule comprising the single chain Fv antibody of claim 4, said molecule being selected from the group consisting of a diabody, a tetrabody, an immunotoxin, a recombinantly produced IgG, Fab, Fab′, F(ab′)₂, F(v), scFv, scFv₂, scFv-Fc, minibody, a bispecific antibody, an Affibody®, and a peptabody.

6. The single chain Fv antibody of claim 4 having immunospecificity for HER3 or EGFR, wherein said antibody comprises an amino acid sequence selected from the group consisting of at least one of SEQ ID NOs:1-19 and 23-41.

7. The single chain Fv antibody of claim 6 which is produced recombinantly.

8. The antibody of claim 1, wherein said antibody is conjugated to a radioisotope.

9. The antibody of claim 12, wherein said radioisotope is selected from the group consisting of ¹⁸F, ¹²⁴I, ⁸⁹Zr, ⁹⁰Y, ¹⁷⁷Lu, ¹³¹I, and ¹⁸⁶ Re.

10. A composition comprising an antibody of claim 1 and a pharmaceutically acceptable carrier.

11. The composition as claimed in claim 10, wherein said antibody is conjugated to at least one molecule selected from the group consisting of a radioisotope, a detectable label, a toxin, a magnetic bead, a pro-drug, a pro-drug converting enzyme, and a chemotherapeutic agent.

12. The composition of claim 10, wherein said antibody is monoclonal.

13. The composition of claim 10, wherein said antibody is a single chain Fv antibody.

14. The composition as claimed in claim 11, wherein said molecule is a radioisotope selected from the group consisting of ¹⁸F, ¹²⁴I, ⁸⁹Zr, ⁹⁰Y, ¹⁷⁷Lu, ¹³¹I, and ¹⁸⁶Re.

15. The composition as claimed in claim 11, wherein said molecule is a toxin selected from the group consisting of maytansanoid, auristatin, saprin, ricin, abrin, ethidium bromide, diptheria toxin, Pseudomonas exotoxin, PE40, PE38, saporin, gelonin, RNAse, protein nucleic acids (PNAs), ribosome inactivating protein (RIP), type-1 or type-2, pokeweed anti-viral protein (PAP), bryodin, momordin, and bouganin.

16. The composition of claim 13, wherein said scFv has immunospecificity for HER3 or EGFR, wherein said antibody comprises an amino acid sequence selected from the group consisting of at least one of SEQ ID NOs:1-19 and 23-41.

17. A method for treating cancer, comprising administering to a patient in need thereof a therapeutically effective amount of the composition of claim 16.

18. The method of claim 18, wherein said cancer is selected from the group consisting of breast, squamous cell carcinoma of the head and neck, prostate, cervical, ovarian, testicular, and pulmonary cancers.

19. The method of claim 19, further comprising administration of at least one chemotherapeutic agent.

20. A vector comprising a nucleic acid sequence encoding the single chain Fv antibody of claim 6.

21. A host cell transformed with the vector of claim 20.

22. A method for imaging cancer in a patient comprising administering to a patient an antibody of claim 1, said antibody optionally comprising a detectable label.

23. A method for imaging cancer in a patient comprising administering to a patient a single chain Fv antibody of claim 6, said antibody optionally comprising a detectable label.

24. The method of claim 22, wherein said detectable label comprises a radioisotope.
